(19) United States
(12) Patent Application Publication
(10) Pub. No.: US 2016/0310725 A1
(43) Pub. Date: Oct. 27, 2016

(54) PADDLE LEAD DELIVERY TOOLS

(71) Applicant: PACESETTER, INC., Sylmar, CA (US)

(72) Inventors: Alan De La Rama, Cerritos, CA (US); Vivian Tran, Laguna Nigel, CA (US); Cary Hata, Irvine, CA (US)

(21) Appl. No.: 15/040,543

(22) Filed: Feb. 10, 2016

Related U.S. Application Data
(60) Provisional application No. 62/152,622, filed on Apr. 24, 2015.

(51) Int. Cl. A61N 1/05; A61B 17/34

(52) U.S. Cl. CPC A61N 1/0553; A61B 17/3468

(57) ABSTRACT

Implementations described and claimed herein provide apparatuses, systems, and methods for paddle lead implantation. In one implementation, a delivery tool for paddle lead implantation includes a hub having a handle port and an insertion port. A handle is engaged to the handle port of the hub and extends proximally from the hub. A sheath extends distally from the hub. The sheath includes a lumen extending through an elongated body from a proximal end to a distal tip. The insertion port includes a port surface configured to collapse a paddle lead for passage into the lumen of the sheath.

### CROSS REFERENCE TO RELATED APPLICATIONS

This application claims the benefit of U.S. Provisional Application Ser. No. 62/152,622, filed Apr. 25, 2015.

### TECHNICAL FIELD

Aspects of the present disclosure relate to apparatuses, systems, and methods for deploying implantable medical devices and more particularly to delivery tools for implanting paddle leads for electrical stimulation of nerve or tissue in a patient.

### BACKGROUND

Medical conditions, such as chronic pain, may be treated through the application of electrical stimulation. For example, Spinal Cord Stimulation (SCS) involves driving an electrical current into particular regions of the spinal cord to induce paresthesia, which is a subjective sensation of numbness or tingling in a region of the body associated with the stimulated spinal cord region. Paresthesia masks the transmission of chronic pain sensations from the afflicted regions of the body to the brain, thereby providing pain relief to the patient. Typically, an SCS system delivers electrical current through electrodes implanted along the dura layer surrounding the spinal cord. The electrodes may be carried, for example, by a paddle lead, which has a paddle-like configuration with the electrodes arranged in one or more independent columns on a relatively large surface area. Paddle leads are generally delivered into the affected spinal tissue through a laminectomy, involving the removal of laminar vertebral tissue to allow access to the dura layer and positioning of the paddle lead. Conventional delivery of paddle leads thus generally requires large incisions and substantial removal of lamina, resulting in trauma to the patient and longer procedure time. As such, there is a need for apparatuses, systems, and methods for delivering large, multi-electrode paddle leads in a minimally invasive surgical approach with minimal vertebral displacement. It is with these observations in mind, among others, that various aspects of the present disclosure were conceived and developed.

### SUMMARY

Implementations described and claimed herein address the foregoing problems, among others, by providing apparatuses, systems, and methods for paddle lead implantation. In one implementation, a delivery tool for paddle lead implantation includes a hub having a handle port and an insertion port. A handle is engaged to the handle port of the hub and extends proximally from the hub. A sheath extends distally from the hub. The sheath includes a lumen extending through an elongated body from a proximal end to a distal tip. The insertion port includes a port surface configured to collapse a paddle lead for passage into the lumen of the sheath.

In another implementation, a hub has a body extending from a proximal surface to a distal surface. A sheath receiver is defined in the body of the hub. A sheath is engaged to the sheath receiver. The sheath extends distally from the hub and includes a lumen extending through an elongated body from a proximal end to a distal tip. An insertion port extends through the body of the hub. The insertion port includes a port surface configured to collapse the paddle lead for passage into the lumen of the sheath.

In another implementation, a paddle lead is received at a first profile of an insertion port, which extends from a proximal surface of a hub to a distal edge of the hub. The first profile is defined in the proximal surface. The paddle lead is collapsed using a port surface of the insertion port. The port surface transitions the paddle lead from the first profile to a second profile at the distal edge. The first profile is different than the second profile and the second profile matches a sheath profile of a proximal end of a sheath. The sheath has a lumen extending from the proximal end to a distal tip. The paddle lead is advanced through the lumen of the sheath through the distal tip.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

FIG. 1 shows an example paddle lead deployment system with a needle inserted into epidural space of a patient.

FIG. 2 illustrates the paddle lead deployment system of FIG. 1 with a guide wire inserted through the needle into the epidural space of the patient.

FIG. 3 illustrates the paddle lead deployment system of FIG. 2 with a delivery tool inserted over the guide wire into the epidural space of the patient.

FIG. 4 shows the paddle lead deployment system of FIG. 3 with an inner penetrator being removed from a sheath of the delivery tool.

FIG. 5 illustrates the paddle lead deployment system of FIG. 4 with a paddle lead being inserted through the sheath of the delivery tool into the epidural space of the patient.

FIG. 6 illustrates the paddle lead implanted and the delivery tool of the paddle lead deployment system of FIG. 5 being removed from the epidural space of the patient.

FIG. 7 depicts an isometric view of an example delivery tool with a handle extension and a distal tip access.

FIG. 8 is a proximal perspective view of the delivery tool of FIG. 7.

FIGS. 9A-9C show a side view, a proximal view, and a perspective distal view, respectively, of an example sheath of the delivery tool of FIG. 7.

FIG. 10 illustrates a distal perspective view of the delivery tool of FIG. 7 with the sheath removed from a hub for clarity.

FIG. 11 is a proximal view of the hub of the delivery tool of FIG. 7.

FIG. 12 is a detailed side cross-sectional view of the delivery tool of FIG. 7 illustrating interfacing between the sheath, hub, and handle.

FIG. 13 illustrates another example of a delivery tool with a handle extension and a distal tip access, shown with the handle pivoting between a plurality of positions and the distal tip closed.

FIG. 14 shows the delivery tool of FIG. 13 with the distal tip expanded as a collapsible paddle lead exits the delivery tool.

FIG. 15 depicts an isometric view of an example delivery tool with a fixed sheath.

FIG. 16 is a proximal perspective view of the delivery tool of FIG. 15.

FIGS. 17A-17C show a side view, a first cross-sectional view, and a second cross-sectional view, respectively, of an example sheath of the delivery tool of FIG. 15.

FIG. 18 illustrates a distal perspective view of the delivery tool of FIG. 15 with the sheath removed from a hub for clarity.

FIG. 19 is a proximal perspective view of the hub of the delivery tool of FIG. 15.

FIG. 20 is a detailed side cross-sectional view of the delivery tool of FIG. 15 illustrating interfacing between the sheath and the hub.

FIG. 21 depicts an isometric view of an example delivery tool with a malleable sheath.

FIG. 22 is a proximal perspective view of the delivery tool of FIG. 21.

FIGS. 23A-23C show a side view, a proximal view, and a distal perspective view, respectively, of an example sheath of the delivery tool of FIG. 21.

FIG. 24 illustrates a distal perspective view of the delivery tool of FIG. 21 with the sheath removed from a hub for clarity.

FIGS. 25A-25B show a distal view and a proximal view, respectively, of the hub of the delivery tool of FIG. 21.

FIG. 26 is a detailed side cross-sectional view of the delivery tool of FIG. 21 illustrating interfacing between the sheath and the hub.

FIG. 27 illustrates an isometric view of a sheath with malleable spines and a coil.

FIG. 28 shows a detailed view of a proximal end of the sheath of FIG. 27.

FIG. 29 illustrates a detailed cross-sectional view of the sheath of FIG. 27.

FIG. 30 is an isometric view of an example hub with finger loops.

### DETAILED DESCRIPTION

Aspects of the present disclosure involve apparatuses, systems, and methods for paddle lead implantation. Generally, a percutaneous delivery tool deploys a paddle lead into the epidural space of a patient using a minimally invasive surgical approach with minimal vertebral displacement. The delivery tool is tracked into position over a guide wire placed in the epidural space. Once the delivery tool is in position, the paddle lead is collapsed and inserted into a lumen of the delivery tool. The paddle lead is advanced through the lumen of the delivery tool into position in the epidural space. The delivery tool is then removed, leaving the paddle lead in position.

In one aspect, the delivery tool includes a hub configured to facilitate insertion of the paddle lead into a smaller profile sheath for deployment into a target location in the epidural space. The hub automatically collapses the paddle lead upon insertion and directs the collapsed paddle lead into a proximal end of the sheath. The collapsed paddle lead is advanced through a lumen of the sheath until it exits through a distal end into position in the epidural space. The distal end may include a soft atraumatic tip that remains in a closed position and moves to an open position as the paddle lead exits. The sheath may have a varying profile shape to accommodate the entry and exit of the paddle lead while maintaining structural support during the procedure. To facilitate maneuvering of the sheath within varying spinal anatomy to access the target location in the epidural space through a small incision, the sheath may be made from a malleable material, include a fixed curve, and/or include malleable spines, and the hub may include a handle extension. The sheath may further include a distal coil to prevent kinking and/or peel-away or split after placement of the paddle lead.

As such, the apparatuses, systems, and methods disclosed herein involve a smaller incision and minimal vertebral displacement, thereby increasing safety, reducing trauma to the patient, minimizing damage to the dura and adjacent tissues, and decreasing procedure time, among other advantages.

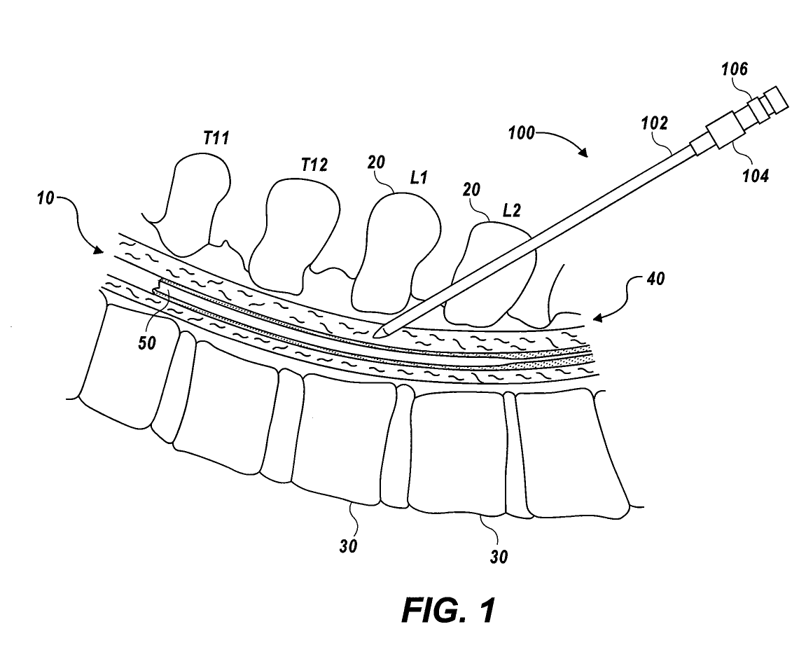

For a detailed description of an example paddle lead deployment system 100, reference is made to FIGS. 1-6. In one implementation, a target location in epidural space 10 of a patient is chosen for positioning a paddle lead to deliver SCS treatment. The target location may be selected, for example, using fluoroscopy. Referring to FIG. 1, in one implementation, the paddle lead deployment system 100 includes a needle 102, which is inserted through a small incision, for example, between spinous processes 20 of two vertebrae 30. The needle 102 is advanced through subcutaneous tissue and ligamentum flavum 40 of the spine into the epidural space 10 along the spinal cord 50. In one implementation, the needle 102 is inserted at an angle, for example, between approximately 35° to 45°. Following entry of the needle 102 into the epidural space 10, an inner portion 106 (e.g., a stylet) is removed from a proximal end 104 of the needle 102.

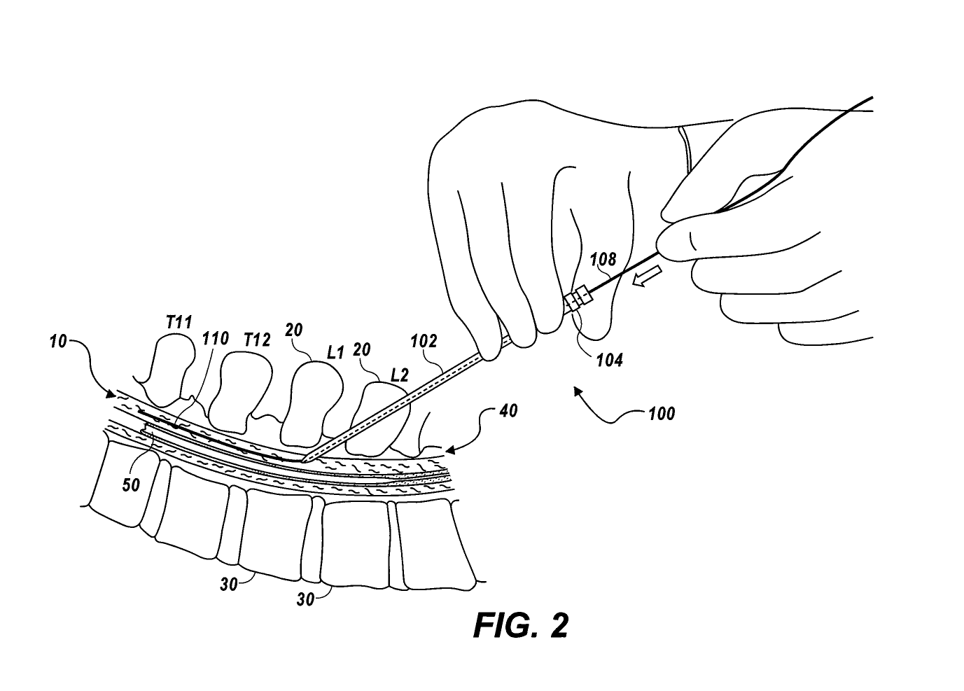

Turning to FIG. 2, in one implementation, after removing the inner portion 106 from the needle 102, a guide wire 108 is inserted through the needle 102 into the epidural space 10. Fluoroscopy may be used to verify a position of a distal end 110 of the guide wire 108 in the target location of the epidural space 10. Once the distal end 110 of the guide wire 108 is positioned, the needle 102 is removed.

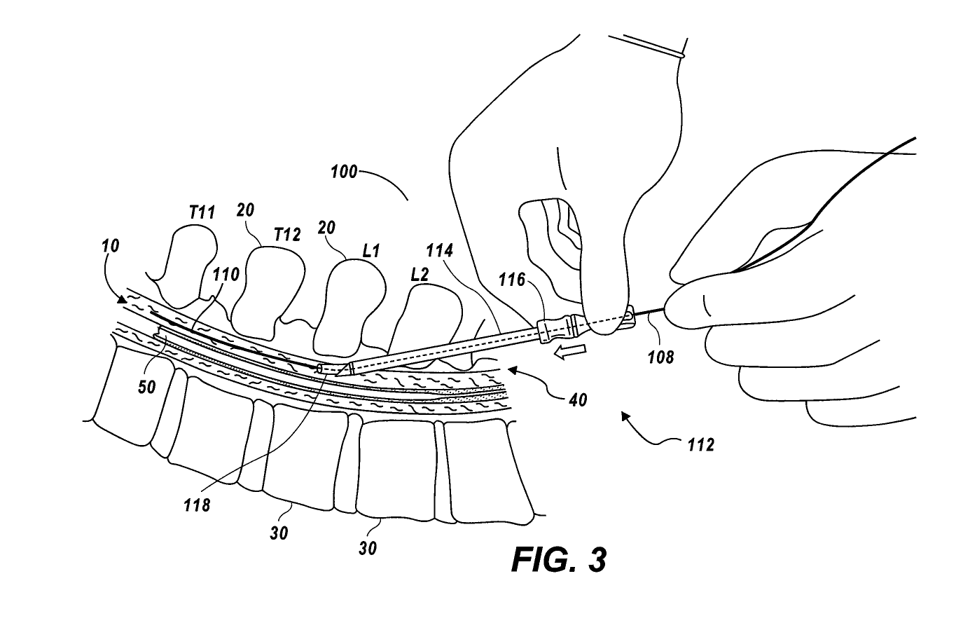

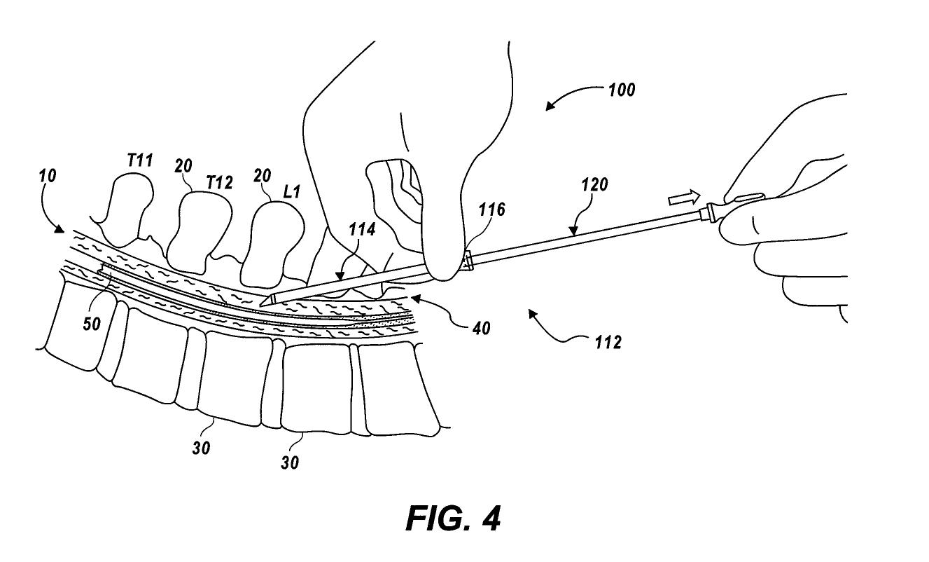

As shown in FIG. 3, a delivery tool 112 having a sheath 114 extending from a hub 116 is deployed over the guide wire 108 into the epidural space 10. The hub 116 may include a directional indicator to assist maneuvering of the delivery tool 112 during deployment. In one implementation, the delivery tool 112 is inserted at an angle, for example, between approximately 35° to 45°.The delivery tool 112 provides a minimal entrance into the epidural space 10, with minimal vertebral displacement. In one implementation, a dilator 118 extends through a distal tip of the sheath 114 from an inner penetrator 120, permitting the delivery tool 112 to pass easily over the guide wire 108 without creating a false passage in an undesirable location of the patient anatomy. The dilator 118 may further provide indication to the surgeon of contact with the ligamentum flavum 40. Once the delivery tool 112 penetrates the ligamentum flavum 40, the guide wire 108 is removed, leaving the sheath 114 positioned in the epidural space 10. As shown in FIG. 4, in one implementation, the inner penetrator 120 is also removed.

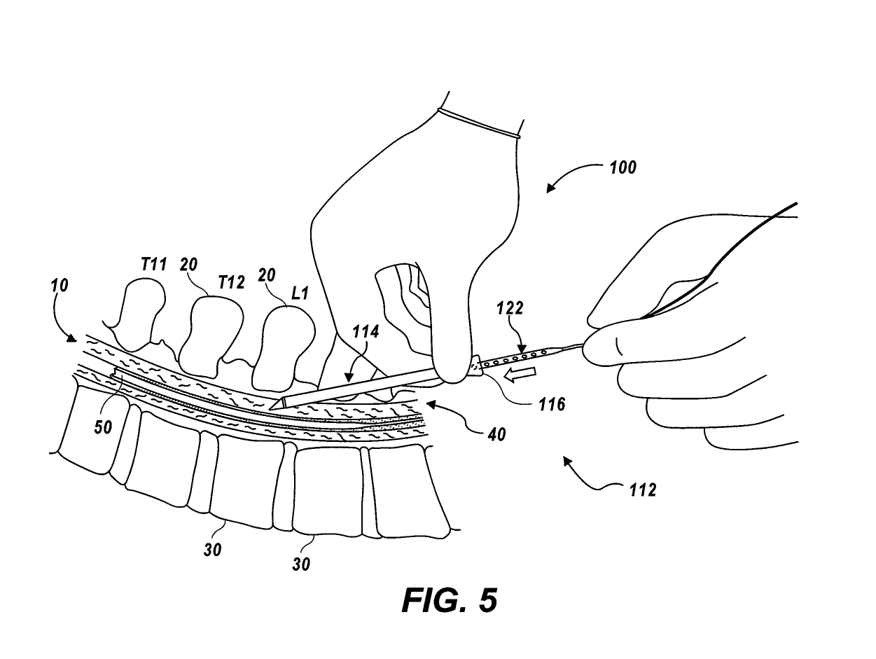

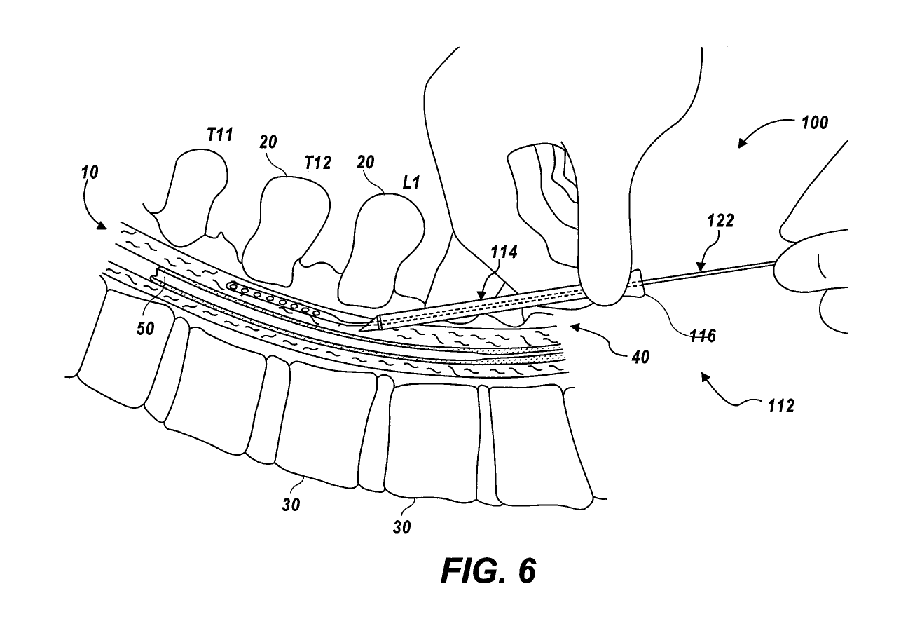

Referring to FIGS. 5 and 6, a paddle lead 122 is inserted through a lumen of the sheath 114 into the target location at an optimal vertebral level in the epidural space 10. The sheath 114 is then removed, leaving the paddle lead 122 in the epidural space 10. The paddle lead 122 may be manipulated to achieve a desired therapeutic effect. In one implementation, the paddle lead 122 is secured by suturing it to a spinous process (e.g., one of the spinous processes 20).

In one implementation, the delivery tool 112 is steerable in a plurality of directions (e.g., 2-4 directions) to assist with positioning the paddle lead 122 in the epidural space 10. The steering may be achieved by displacement of wires extending through the lumen of the sheath 114 using the hub 116. In one implementation, an insertion port is disposed along the sheath 114. A profile of the insertion port may have a variety of shapes, including, without limitation, circular, elliptical, obround, rectangular, angled, contoured, and/or the like. A shape and size of the profile of the insertion port is configured to collapse the paddle lead 122 for passage into the lumen of the sheath 114. Inserting the paddle 122 in a controlled orientation influences the orientation in which the paddle 122 exits the sheath 114 into the epidural space 10. In one implementation, a distal tip of the sheath 114 has an elliptical profile shape, providing additional control of the orientation of the paddle lead 122 as it exits the sheath 114.

The materials and build configuration of the sheath 114 may be modified to adjust the flexibility and kink resistance of the lumen. For example, the sheath 114 of the delivery tool 112 may have a liner constructed of materials, including, but not limited to, a thermoplastic elastomer (e.g., polyether block amide), a synthetic polymer (e.g., nylon), polytetrafluoroethylene (PTFE), and SST braid. In one implementation, at least a portion of the sheath 114 is loaded with BaSO4 or similar substance for radiopacity, and the distal tip of the sheath 114 may include a platinum iridium marker band for additional visualization. The dilator 118 may similarly be constructed of LDPE, HDPE, BaSO4, and/or the like for radiopacity. It will be appreciated that the dilator 118 may be constructed of various other polymers or materials to modify flexibility, hardness, and other performance factors of the delivery tool 112. In one implementation, the dilator 118 comprises a soft durometer polyether block amide tip.

The size and shape of the sheath 114 may vary depending, among other factors, on a width of the paddle lead 122 and a length of the lead. In one implementation, the sheath 114 has an inner diameter of approximately 11 French Units (approximately 0.19 inches), an outer diameter of approximately 14.5 French Units (approximately 0.15 inches), and a length of approximately 25 centimeters, and the dilator 118 has an inner diameter of approximately 0.06 inches. The sheath 114 may be configured to permit splitting by a splitting tool having a handle with a cutting surface (e.g., a razor blade).

In another implementation, the delivery tool 112 is steerable using one or more wires extending through one or more lumens of the sheath 114 and/or via a curved wire configured for advancement and retraction. For example, the sheath 114 may include a primary lumen for receiving the paddle lead 122 and one or more secondary lumens through which wires may extend for steering. The secondary lumens may be smaller in size relative to the primary lumen and/or positioned adjacent to the primary lumen, for example, in a surface of the hub 116. In one implementation, the sheath 114 is constructed of a co-extruded lumen having an elliptical shape. It will be appreciated that other shapes may be used, including, but not limited to, circular, obround, angled, contoured, and/or the like. The sheath 114 may include a plurality of sections connected together for variable stiffness. The sections may be connected through heat bonding, using an adhesive, mechanical connection, and/or the like. In one implementation, the sheath 114 is reinforced with metal or similar material in one or more areas of the sheath 114 for variable stiffness.

In yet another implementation, the delivery tool 112 is non-steerable. In one implementation, an insertion port is disposed along the sheath 114. A profile of the insertion port may have a variety of shapes, including, without limitation, circular, elliptical, obround, angled, contoured, and/or the like. A shape and size of the profile of the insertion port is configured to collapse the paddle lead 122 for passage into the lumen of the sheath 114. Inserting the paddle 122 in a controlled orientation influences the orientation in which the paddle 122 exits the sheath 114 into the epidural space 10. In one implementation, the delivery tool 112 includes a second sheath, which adds increased stiffness to at least a portion of the sheath 114, facilitating access to the epidural space 10 and advancement of the sheath 114. The sheath 114 may be constructed in a fixed curve configuration. In this implementation, the sheath 114 is inserted with the curve oriented upwards, and rotating of the sheath 114 moves the distal tip of the sheath 114 to navigate to a desired location and orientation in the epidural space 10. The sheath 114 may include a lumen comprising a flat braid providing sufficient torque response for control of the orientation.

The materials and build configuration of the sheath 114 may be modified to adjust the flexibility and kink resistance of the lumen. For example, the sheath 114 of the delivery tool 112 may have a liner constructed of materials, including, but not limited to, a thermoplastic elastomer (e.g., polyether block amide), a synthetic polymer (e.g., nylon), polytetrafluoroethylene (PTFE), and SST braid. In one implementation, at least a portion of the sheath 114 is loaded with BaSO4 or similar substance for radiopacity, and the distal tip of the sheath 114 may include a polymer ring loaded with tungsten for additional visualization. The polymer ring may be proximal to a soft durometer tip of the sheath 114. The dilator 118 may similarly be constructed of LDPE, HDPE, BaSO4, and/or the like for radiopacity.

The size and shape of the sheath 114 and the secondary sheath may vary depending, among other factors, on a width of the paddle lead 122 and a length of the lead. In one implementation, the sheath 114 has an inner diameter of approximately 11 French Units, an outer diameter of approximately 0.18 inches, and a length of approximately 25 centimeters, and the secondary sheath has an outer diameter of approximately 0.20 inches.

In one implementation, the delivery tool 112 comprises the hub 116, the sheath 114 with a soft atraumatic distal tip section, and a handle extension for delivering the collapsible paddle lead 122 into the epidural space 10. The handle extension may include a handle body extending from the hub 116 and mounted on a pivot, permitting movement of the handle body between a plurality of positions to facilitate maneuvering of the delivery tool 112 within a small incision. In one implementation, the hub 116 includes an insertion port configured to receive the paddle lead 122 flat upon insertion to facilitate advancement into a proximal end of the sheath 114. The insertion port may have a variety of profile shapes and sizes configured for a smooth transition of the paddle lead 122 from the hub 116 into the lumen of the sheath 114.

To facilitate the deployment of the paddle lead 122 from the lumen of the sheath 114 into the target location in the epidural space 10, the sheath 114 may have a profile shape that varies along a length of the sheath 114. For example, the sheath 114 may have a profile shape that varies along the length of the sheath 114 from circular to elliptical. A circular profile shape provides structural support during the procedure, and the elliptical profile shape begins unfolding the collapsed paddle lead 122 for deployment from the distal tip into the target location in the epidural space 10. In one implementation, the sheath 114 is constructed with a rigid material, such as a rigid polymer and/or an elastomeric polymer with braiding for support. Such a rigid material may reduce cost and facilitate manufacturing. In another implementation, the sheath 114 is made from a malleable material, such as stainless steel or other malleable metals, to permit bending of the sheath 114 to an angle configured to accommodate the anatomy of the patient. The sheath 114 may thus have a fixed curve at an angle, including, but not limited to 0°, 15°, 30°, 45°, or the like, or have a flexible distal tip. To minimize trauma to the patient from the delivery tool 112 during the procedure, the sheath 114 may comprise a soft material, such as a lower durometer elastomeric polymer.

Thus, the paddle lead deployment system 100 delivers the paddle lead 122 into the epidural space 10 through a smaller incision and with minimal vertebral displacement, thereby increasing safety, reducing trauma to the patient, minimizing damage to the dura and adjacent tissues, and decreasing procedure time, among other advantages.

As described herein, the paddle lead 122 is inserted into an insertion port, such as a port on the hub 116 and/or along a length of the sheath 114. The insertion port collapses the paddle lead 122 into a collapsed orientation for advancement into the lumen of the sheath 114. It will be appreciated that the paddle lead 122 may be furled and unfurled in other manners. More particularly, in one implementation, edges of the paddle lead 122 are folded in a same direction about the lumen of the sheath 114 and subsequently unfolded for deployment. In another implementation, the edges of the paddle lead 122 are wrapped in a same direction about the lumen of the sheath 114 with rotation of the lumen deploying the paddle lead 122, thereby enabling a smaller profile of the paddle lead 122 during delivery through the delivery tool 112.

In another implementation, each edge of the paddle lead 122 accordions relative to the lumen of the sheath 114, forming a paddle profile with an elongated profile in a first direction and a thin profile in a second direction. The sheath 114 may have a profile matching the paddle profile to maintain an orientation of the paddle lead 122 with electrode surfaces facing a desired direction throughout deployment. As the paddle lead 122 exits the distal tip of the sheath 114, the accordion sides may spring out from the lumen or otherwise unfurl.

In yet another implementation, the paddle lead 122 remains furled until the paddle lead 122 is delivered into the target location distal to the distal tip of the sheath 112. Once the paddle lead 122 is placed in the target location, a release, such as an internal balloon, axial compression, and/or the like, unfurls and deploys the paddle lead 122. In still another implementation, the paddle lead 122 is preloaded into the distal tip of the sheath 114 and delivered to the target location, where the sheath 114 is withdrawn while holding the paddle lead 114 in place, thereby unfurling and deploying the paddle lead 122 in the target location. A distance the paddle lead 122 travels within potentially tortuous pathways of the patient during deployment is thus minimized.

The sheath 114 and/or other features of the delivery tool 112 may be configured to maintain a shape of the paddle lead 122 during and after deployment. In one implementation, the sheath 114 is made with a NiTi structure. In another implementation, the sheath 114 utilizes elastic polymers, such as silicone, and/or polymer composites. For example, the sheath 114 may be a Polyether ether ketone (PEEK) frame encapsulated in silicone. Such materials may simplify assemble of the paddle lead 122 and reduce a risk of shorting between electrodes, as well as delimitation from a polymer body of the paddle lead 122 due to repetitive flexure and/or removal forces. The sheath 114 may further include one or more profile shapes along a length of the sheath 114 configured to facilitate unfurling of the paddle lead 114 during and/or after deployment. An application of a lubricious coating, such as a hydrogel, to the paddle lead 122 may further assist in the unfurling and deployment of the paddle lead 122. The movement of the paddle lead 122 through the lumen of the sheath 114 and elsewhere in the delivery tool 112 during deployment may create undesirable resistance. The lubricious coating thus reduces such resistance, while providing enhanced tactile feedback during deployment.

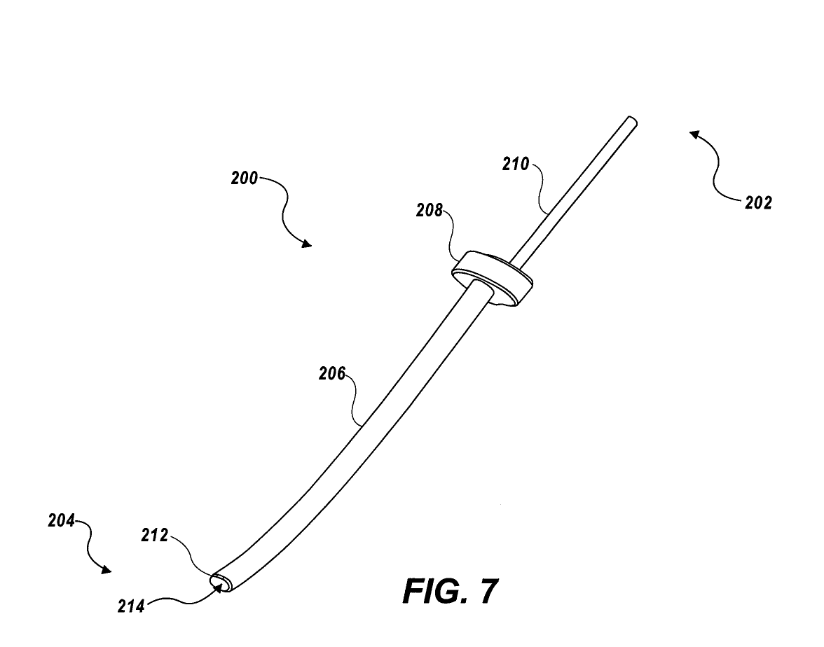

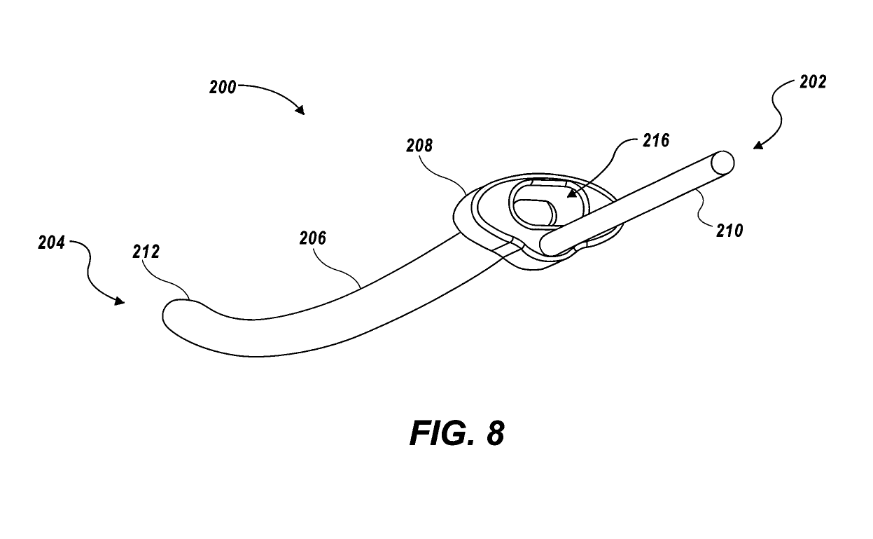

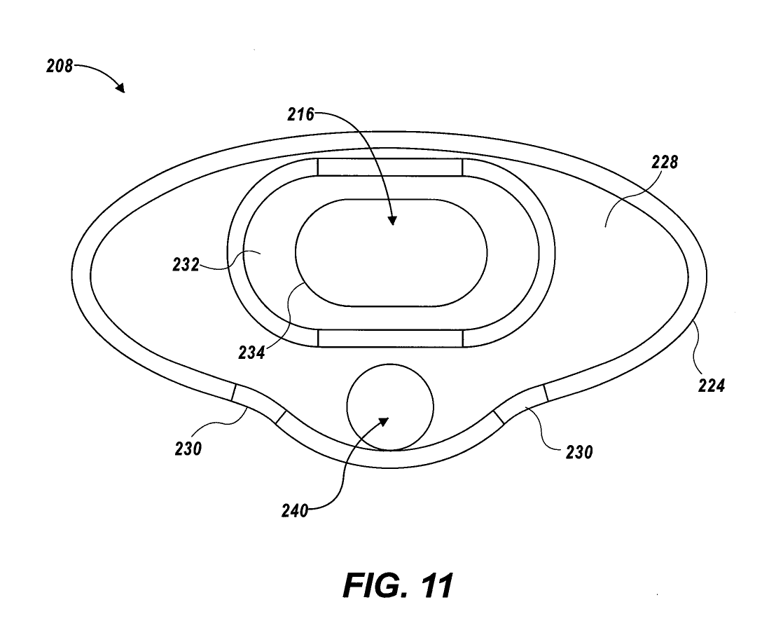

For a detailed discussion of an example delivery tool 200 with a handle extension and a distal tip access, reference is made to FIGS. 7-14. Referring to FIGS. 7 and 8, which show an isometric view and a proximal perspective view of the delivery tool 200, respectively, the delivery tool 200 generally extends between a proximal end 202 and a distal end 204. In one implementation, a sheath 206 extends distally from a hub 208, and a handle 210 extends proximally from the hub 210. The sheath 206 includes a lumen 214 extending distally through a length of the sheath 206 and a distal tip 212. In one implementation, the hub 208 includes an insertion port 216 configured to collapse the paddle lead 122 for passage into the lumen 214. Inserting the paddle 122 in a controlled orientation influences the orientation in which the paddle 122 exits the distal tip 212 into the epidural space 10.

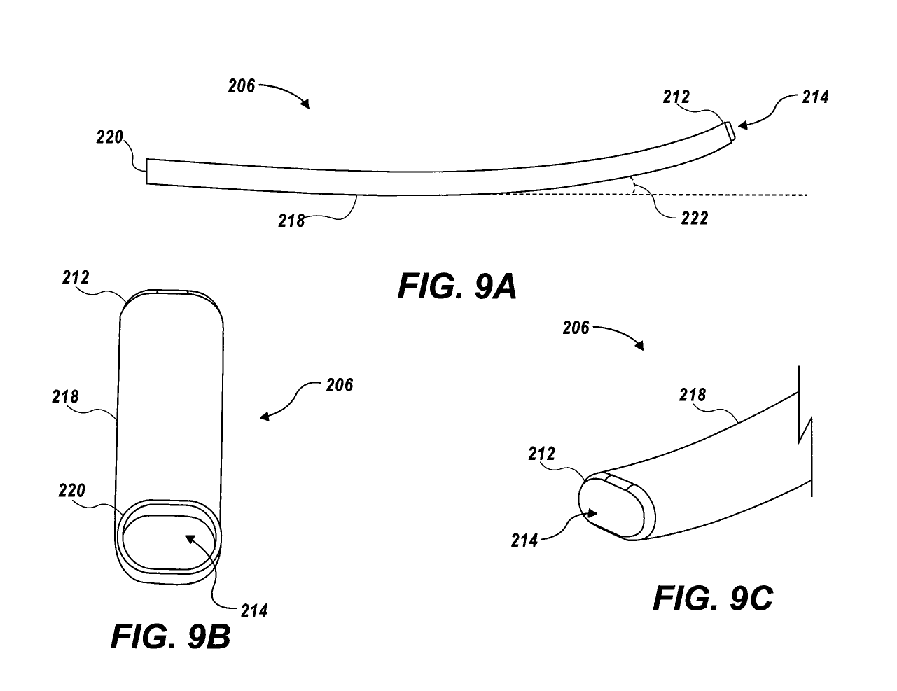

Turning to FIGS. 9A-9C, in one implementation, the sheath 206 includes an elongated body 218 extending between a proximal end 220 and the distal tip 212. As described herein, the elongated body 218 may include a fixed curve or be malleable. In one implementation, the elongated body 218 includes a fixed curve near the distal tip 212 at an angle 222 of approximately 15°, 30°, or 45°. In another implementation, the elongated body 218 is substantially straight. The sheath 206 may have one or more profile shapes along a length of the elongated body 218. In one implementation, the profile shape of the sheath 206 is constant from the proximal end 220 to the distal tip 212. For example, as shown in FIGS. 9B and 9C, the proximal end 220 and the distal tip 212 may each have a profile with an obround shape defined by a pair of opposing lines extending transversely to a length of the elongated body 218 and connected by a pair of opposing semicircles. In another implementation, a profile shape of the sheath 206 at the proximal end 220 is different than a profile shape of the sheath 206 at the distal tip 212. The one or more profile shapes of the sheath 206 may include, without limitation, circular, elliptical, obround, rectangular, angled, contoured, and/or the like.

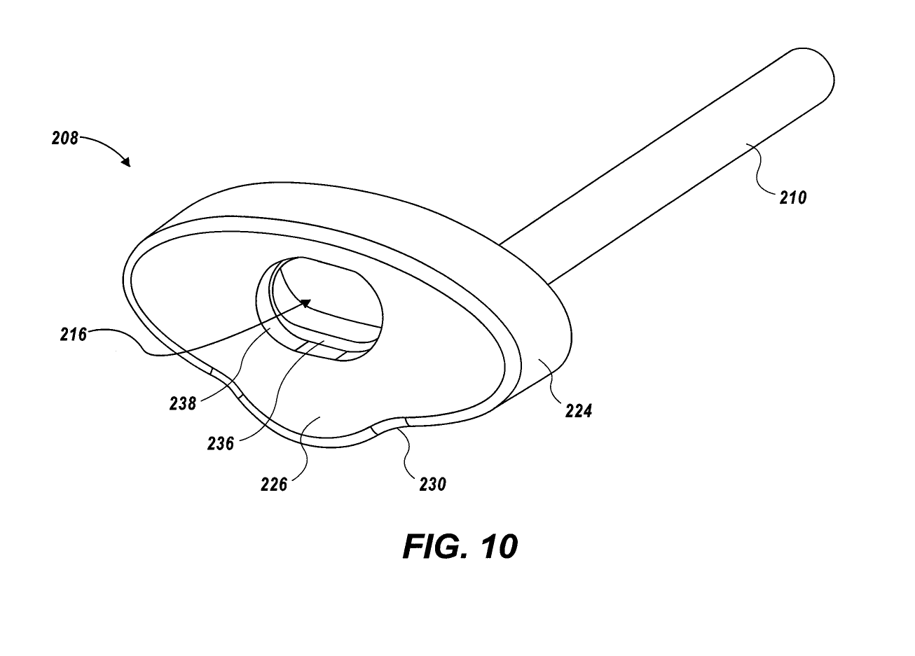

As can be understood from FIGS. 10-12, the insertion port 216 of the hub 208 is configured to facilitate insertion of the paddle lead 122 into the lumen 214 of the sheath 206. In one implementation, the hub 208 includes a body 224 extending between a distal surface 226 and a proximal surface 228. The body 224, the proximal surface 228, and the distal surface 226 may each be a variety of shapes and/or include surface(s) with various textures. In one implementation, the proximal surface 228 and the distal surface 226 are smooth, planar surfaces, and the body 224 is rounded and smooth with one or more indents 230.

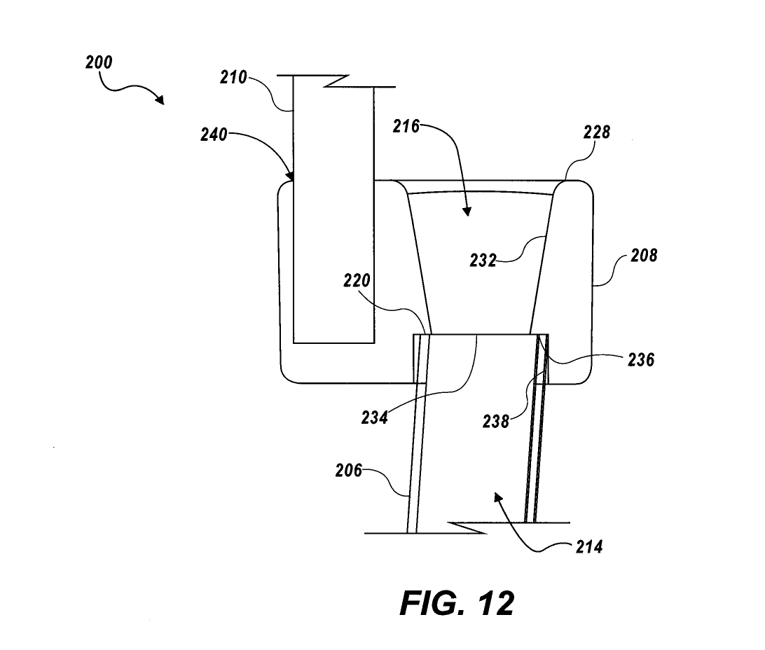

In one implementation, the insertion port 216 extends through the body 224 of the hub 208 from the proximal surface 228 to the distal surface 226. The insertion port 216 is defined by a port surface 232 extending distally from the proximal surface 228 to a distal edge 234. In one implementation, the port surface 232 is angled, such that the insertion port 216 tapers in diameter distally from the proximal surface 228 to the distal edge 234 to match the lumen 214 of the sheath 206. Stated differently, the size and profile shape of the distal edge 234 may match the size and profile shape of the lumen 214 at the proximal end 220 of the sheath 206. To engage the sheath 206, in one implementation, the hub 208 includes a sheath receiver, defined by a shelf 236 extending inwardly toward a center of the insertion port 216 from a receiver surface 238. As shown in FIGS. 10 and 12, the receiver surface 238 extends from the distal surface 226 of the hub 208 to the shelf 236. Once the sheath 206 is engaged to the hub 208, in one implementation, the distal edge 234 of the insertion port 216 and the proximal end 220 of the sheath 206 are coplanar.

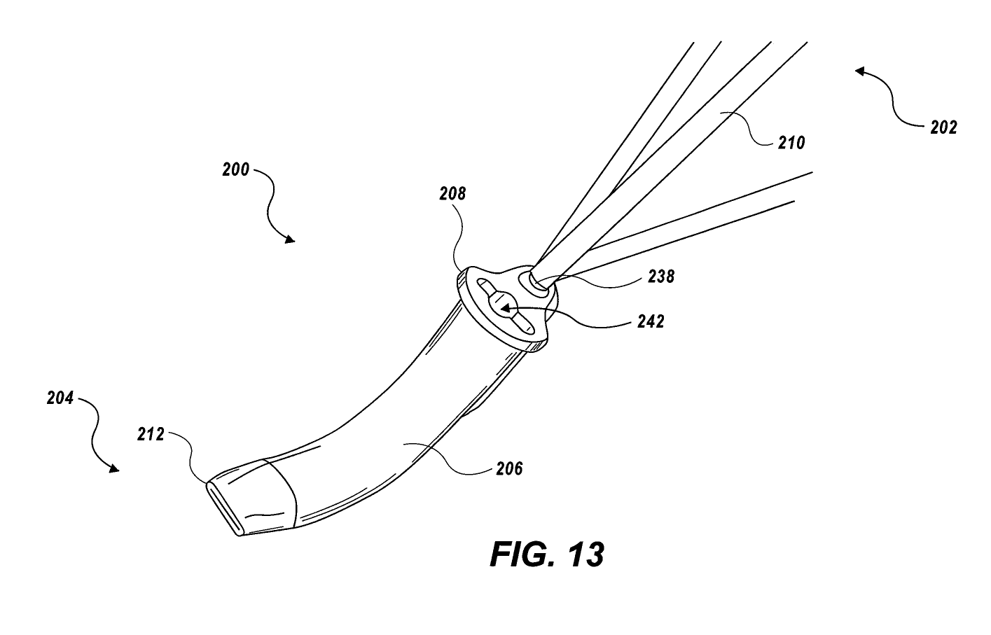

To facilitate maneuvering of the delivery tool 200 within tight spaces in the anatomy of the patient, in one implementation, the hub 208 includes a handle port 240 defined in the body 224 through the proximal surface 228 and configured to engage the handle 210. As can be understood from FIG. 13, the handle port 240 may be configured to pivotally engage the handle 210, such that the handle 210 may be moved to a plurality of positions. In one implementation, the handle 210 terminates in a pivot ball 242 at a distal end, permitting the handle 210 to be pivoted to a plurality of positions.

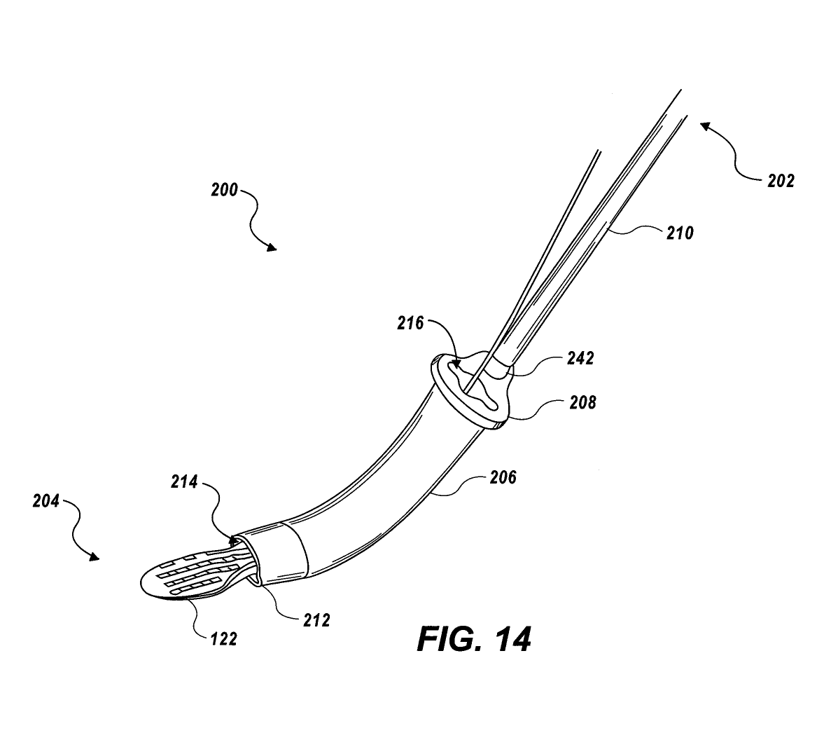

As described herein, the distal tip 212 may be made from a soft atraumatic material. Further, as shown in FIGS. 13 and 14, the distal tip 212 may remain in a closed position until the paddle lead 122 exits the distal tip 212. Stated differently, the distal tip 212 moves from the closed position to an open position upon the paddle lead 122 moving through and exiting the distal tip 212.

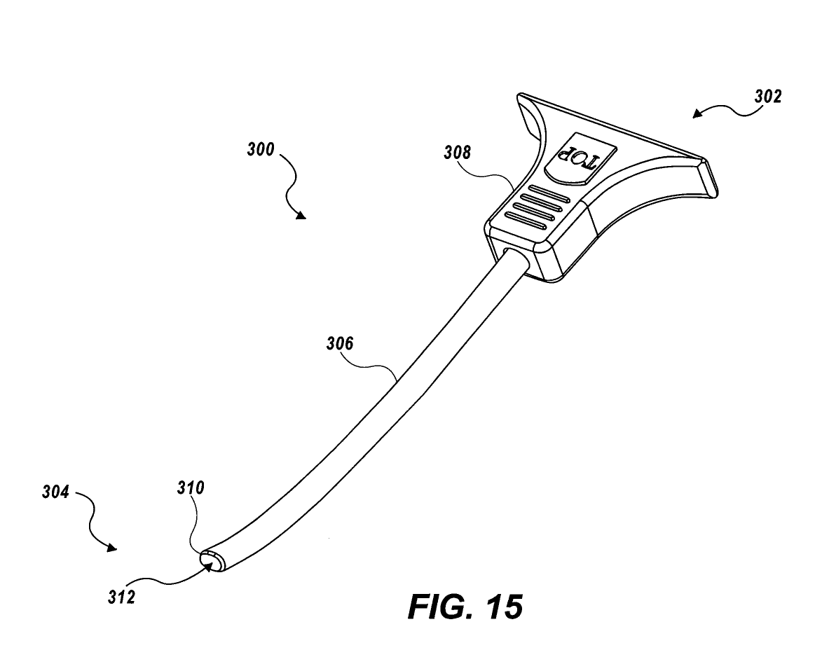

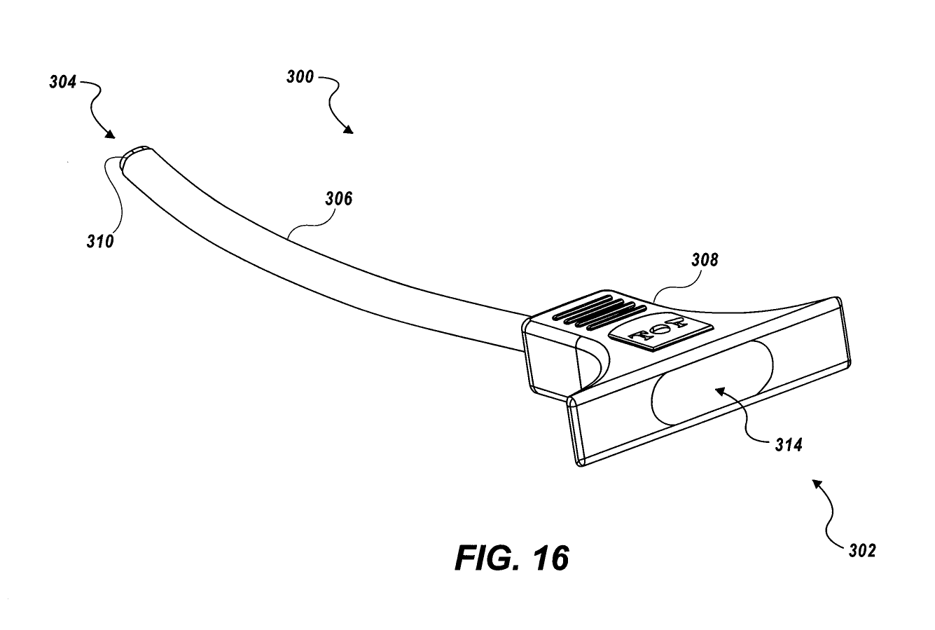

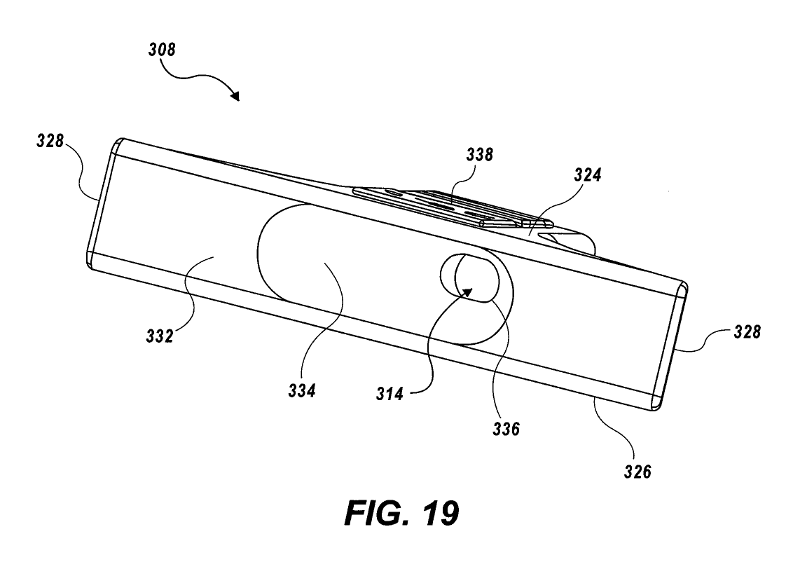

For a detailed discussion of another example delivery tool 300, reference is made to FIGS. 15-20. Referring to FIGS. 15 and 16, which show an isometric view and a proximal perspective view of the delivery tool 300, respectively, the delivery tool 300 generally extends between a proximal end 302 and a distal end 304. In one implementation, a sheath 306 extends distally from a hub 308, which may include a directional indicator to inform maneuvering of the delivery tool 300 during the procedure. The sheath 306 includes a lumen 312 extending distally through a length of the sheath 306 and a distal tip 310. In one implementation, the hub 308 includes an insertion port 314 configured to collapse the paddle lead 122 for passage into the lumen 312. Inserting the paddle 122 in a controlled orientation influences the orientation in which the paddle 122 exits the distal tip 310 into the epidural space 10, as described herein. It will be appreciated that the delivery tool 300 further accommodates the dilator 118 for insertion into the sheath 306 and for the guide wire 108 through a luer port.

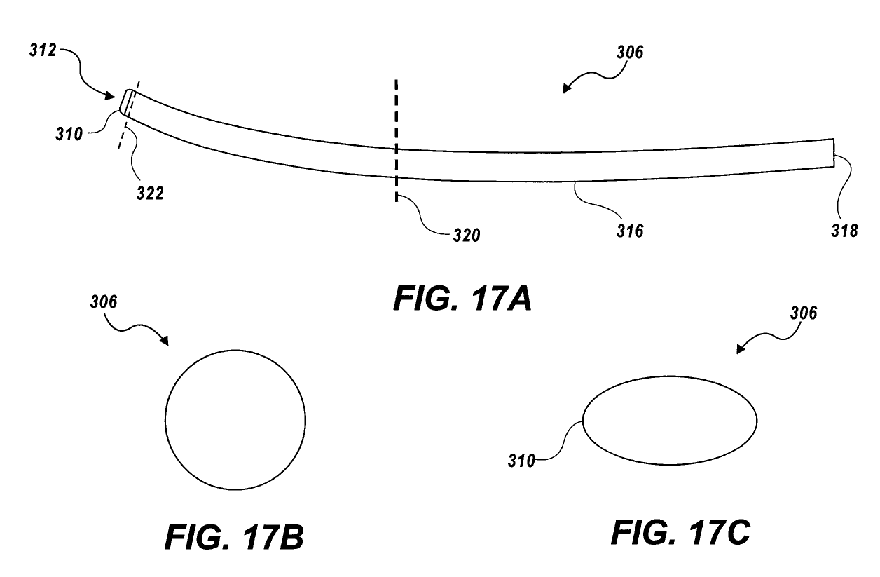

Turning to FIGS. 17A-17C, in one implementation, the sheath 306 includes an elongated body 316 extending between a proximal end 318 and the distal tip 310. As described herein, the elongated body 316 may include a fixed curve or be malleable. In one implementation, the elongated body 316 is a rigid polymer tube having a fixed curve near the distal tip 310. The rigid polymer may be, for example, high density polyethylene or low density polyethylene. In another implementation, the elongated body 316 is substantially straight.

The sheath 306 may have one or more profile shapes along a length of the elongated body 316. In one implementation, the profile shape of the sheath 306 is variable from the proximal end 318 to the distal tip 310. For example, the sheath 306 may have a different profile at a first location 320 proximal to the fixed curve from a second location 322 near the distal tip 310. In one implementation, the first location 320 has a circular profile shape and the second location 322 has an elliptical profile shape. Stated differently, the elongated body 316 has a circular profile shape from the proximal end 318 to the first location 320, where the profile shape of the elongated body 316 transitions into the elliptical profile shape of the second location 322. The transition of the profile shape of the elongated body 316 gradually unfolds the paddle lead 122 as it is advanced towards the distal tip 310. The profile shape of the elongated body 316 at the second location 322 controls an orientation of the paddle lead 122 as it exits the delivery tool 300, thereby facilitating implantation of the paddle lead 122 in the proper orientation at the target location in the epidural space 10, as well as reducing stress to the adjacent tissue. A delivery stage disposed near the distal tip 310 may include a distally extending or projecting lip, ledge, or the like to provide additional control of the deployment of the paddle lead 122.

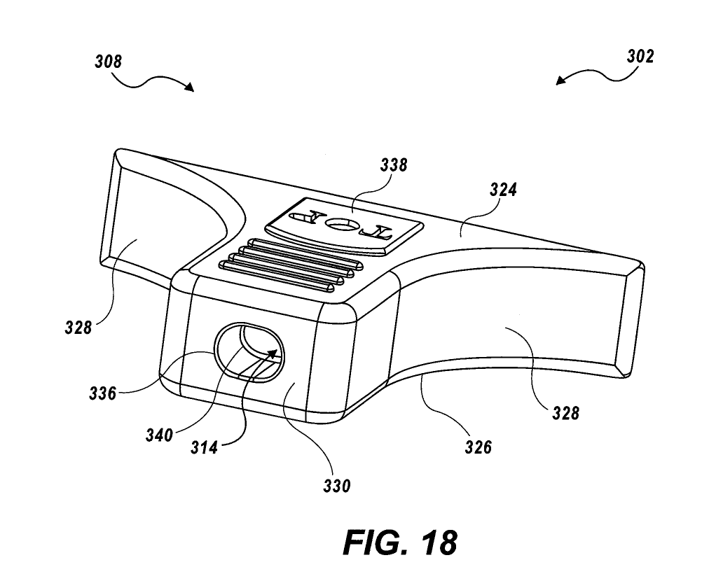

As can be understood from FIGS. 18-20, the insertion port 314 of the hub 308 is configured to facilitate insertion of the paddle lead 122 into the lumen 312 of the sheath 306. In one implementation, the hub 308 includes a body formed by a top surface 324 disposed opposite a bottom surface 326 and connected by a pair of opposing side surfaces 328, a proximal surface 332, and a distal surface 330 disposed opposite the proximal surface 332. It will be appreciated, however, that the body may be a variety of shapes and/or include surface(s) with various textures. In one implementation, the top surface 324 and the bottom surface 326 are smooth, planar surfaces, and the top surface 324 includes a directional indicator 338 identifying the top surface. The directional indicator 338 may include, without limitation, words, graphics, textures, colors, designs, and/or other indicators. In one implementation, the proximal surface 332 has a longer length, extending transverse to a length of the sheath 306, relative to a length of the distal surface 330. A size of the body thus tapers distally along a curve of each the side surfaces 328 from the proximal surface 332 to the distal surface 330. The hub 308 may be configured to split open, as needed, during the procedure.

In one implementation, the insertion port 314 extends through the body of the hub 308 from the proximal surface 332 to the distal surface 330. The insertion port 314 is defined by a port surface 334 extending distally from the proximal surface 332 to a distal edge 336. In one implementation, the port surface 334 is angled, such that the insertion port 314 tapers in diameter distally from the proximal surface 332 to the distal edge 336 to match the lumen 312 of the sheath 306. Stated differently, the size and profile shape of the distal edge 336 may match the size and profile shape of the lumen 312 at the proximal end 318 of the sheath 306. In one implementation, the insertion port 314 has a rectangular shape at the proximal surface 332, and the port surface 334 tapers distally into a circular opening at the distal edge 336 to facilitate a smooth transition of the paddle lead 122 from insertion at the proximal surface 332 into the lumen 312 of the sheath 306.

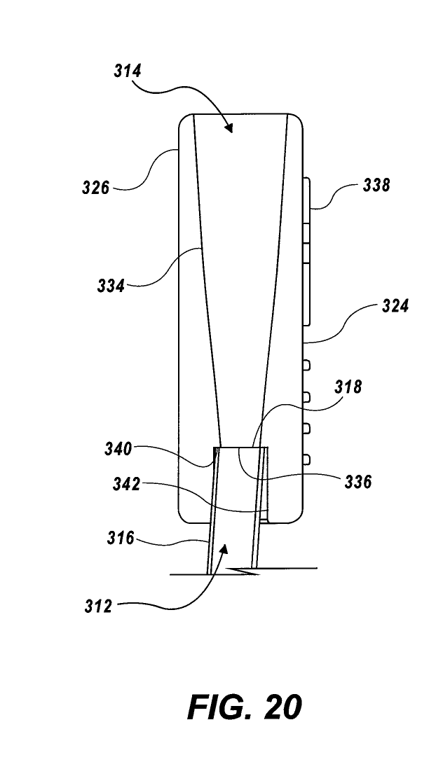

To engage the sheath 306, in one implementation, the hub 308 includes a sheath receiver, defined by a shelf 340 extending inwardly toward a center of the insertion port 314 from a receiver surface 342. As shown in FIGS. 18 and 20, the receiver surface 342 extends from the distal surface 330 of the hub 308 to the shelf 340. Once the sheath 306 is engaged to the hub 308, in one implementation, the distal edge 336 of the insertion port 314 and the proximal end 318 of the sheath 306 are coplanar.

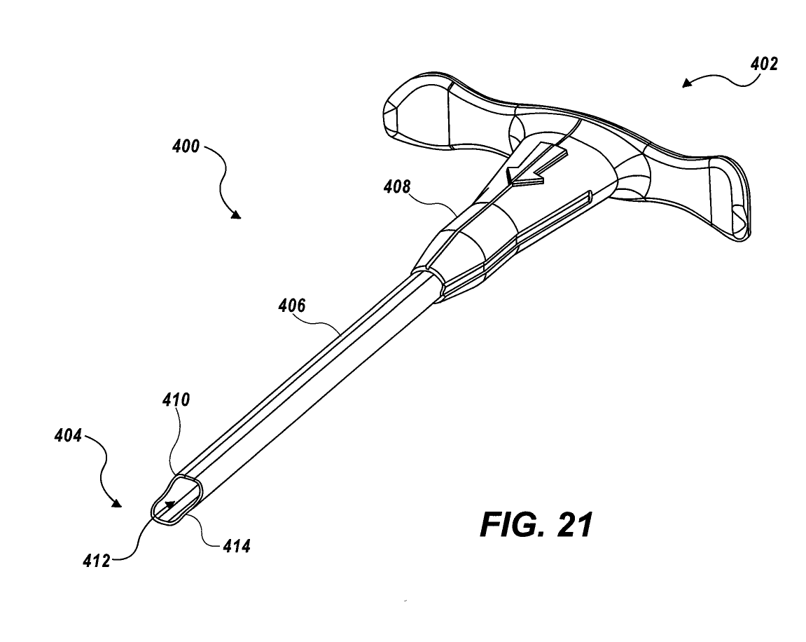

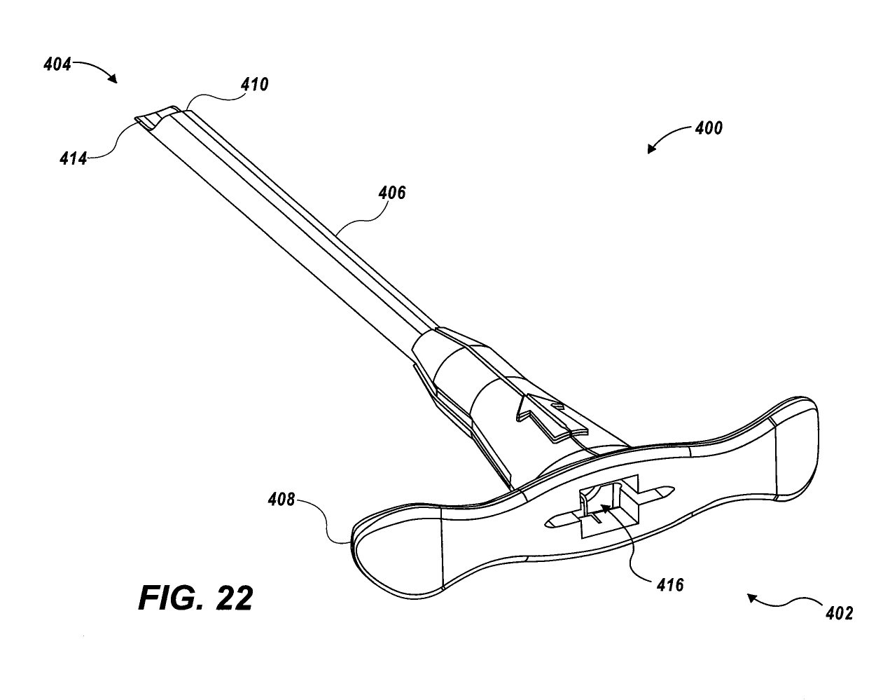

For a detailed discussion of yet another example delivery tool 400, reference is made to FIGS. 21-26. Referring to FIGS. 21 and 22, which show an isometric view and a proximal perspective view of the delivery tool 400, respectively, the delivery tool 400 generally extends between a proximal end 402 and a distal end 404. In one implementation, a sheath 406 extends distally from a hub 408. The sheath 406 includes a lumen 412 extending distally through a length of the sheath 406 and a distal tip 410. A delivery stage 414 disposed near the distal tip 410 may include a distally extending or projecting lip, ledge, or the like to provide additional control of the deployment of the paddle lead 122.

In one implementation, the hub 408 includes an insertion port 416 configured to collapse the paddle lead 122 for passage into the lumen 412. Inserting the paddle 122 in a controlled orientation influences the orientation in which the paddle 122 exits the distal tip 410 into the epidural space 10, as described herein. It will be appreciated that the delivery tool 400 further accommodates the dilator 118 for insertion into the sheath 406 and for the guide wire 108 through a luer port.

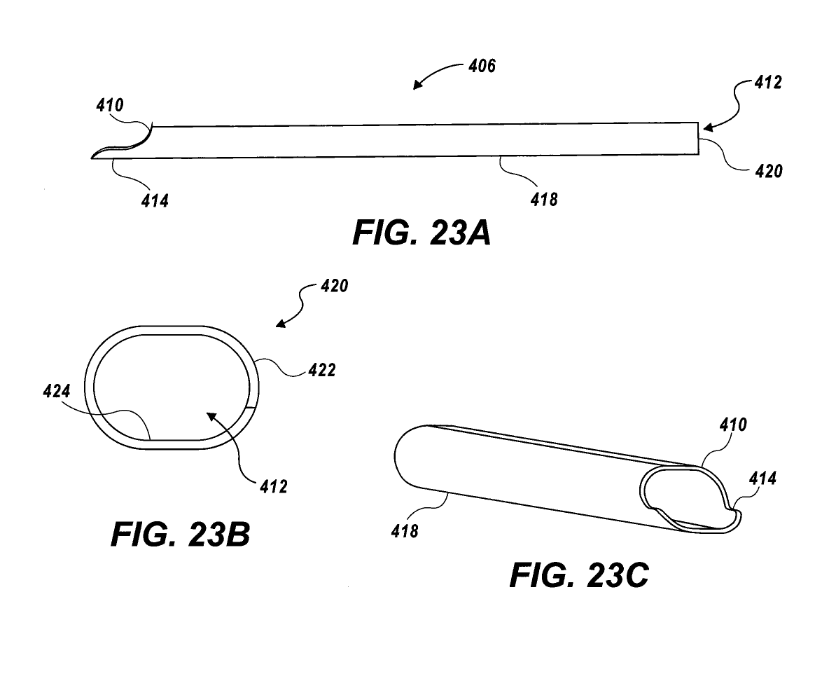

Turning to FIGS. 23A-23C, in one implementation, the sheath 406 includes an elongated body 418 extending between a proximal end 420 and the distal tip 410. As described herein, the elongated body 418 may include a fixed curve or be malleable. In one implementation, the elongated body 418 is a constructed from a malleable material, such as stainless steel, permitting the sheath 406 to be bent to an angle to manually accommodate the varying patient anatomy along the path into the epidural space 10. In another implementation, the sheath 406 is a metal tube comprising varying spiral cuts to achieve different flexibility or stiffness.

In one implementation, the elongated body 418 includes a liner 424 comprising a smooth, low friction polymer, such as tetrafluoroethylene (TFE), Polytetrafluoroethylene (PTFE), or other lubricious material. The liner 424 may further include a hydrophilic coating. The elongated body 418 may include an outer jacket 422 comprising a polymer, such as a polyurethane-silicone mixture.

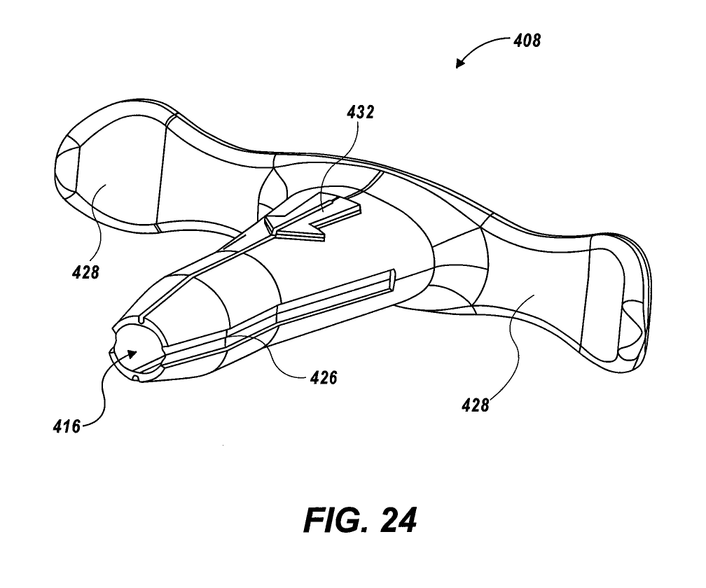

As can be understood from FIGS. 24-26, the insertion port 416 of the hub 408 is configured to facilitate insertion of the paddle lead 122 into the lumen 412 of the sheath 406. The sheath 406 may have one or more profile shapes along a length of the elongated body 418. For example, the profile shape of the elongated body 418 may transition from circular to elliptical, as described herein.

In one implementation, the hub 408 includes a body 426 extending from a grip formed by a proximal surface 430 disposed opposite a grip surface 428. The proximal surface 430 may include a directional indicator 432 to inform maneuvering of the delivery tool 400 during the procedure. The directional indicator 432 may include, without limitation, words, graphics, textures, colors, designs, and/or other indicators.

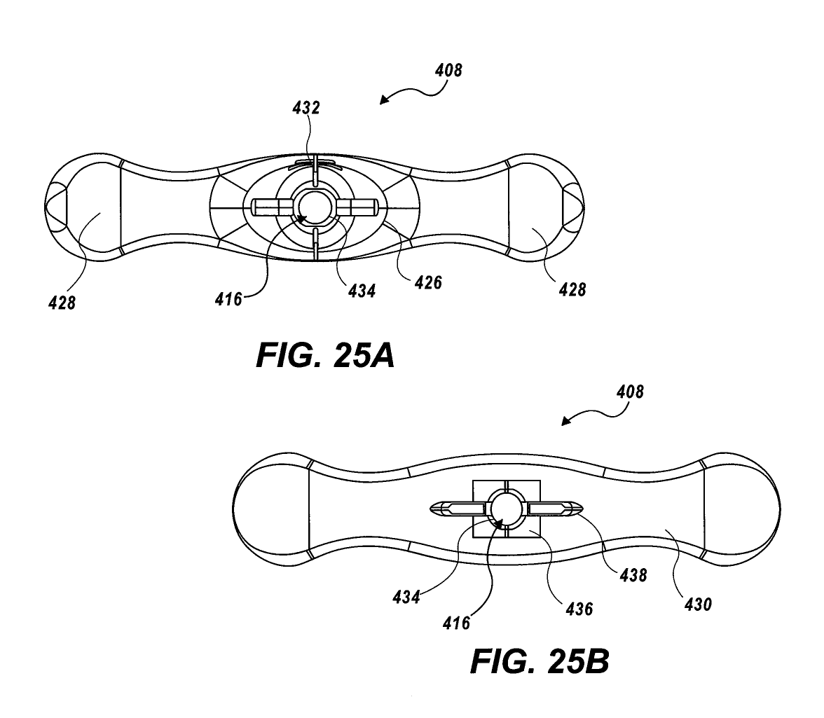

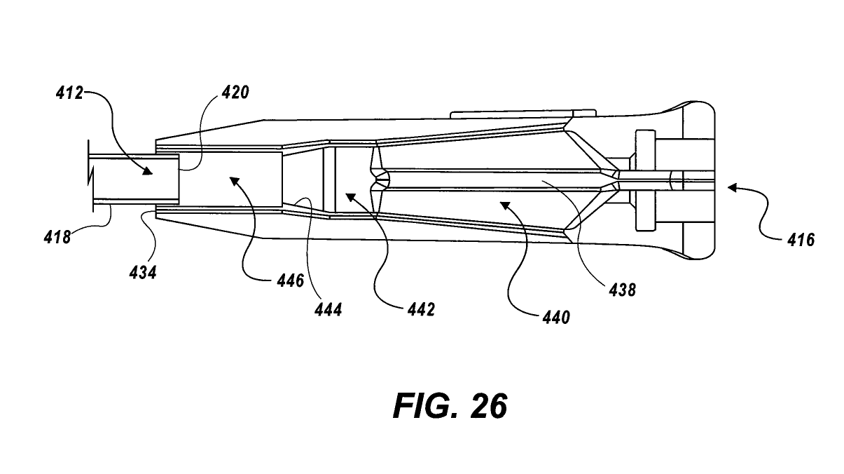

In one implementation, the insertion port 416 extends through the body 426 of the hub 408 from the proximal surface 430 to a distal edge 434. The insertion port 416 may have a varying profile shape to facilitate a smooth transition of the paddle lead 122 from insertion at the proximal surface 430 into the lumen 412 of the sheath 406. As can be understood from FIGS. 25A-26, in one implementation, a profile shape of track 438 of the insertion port 416 at the proximal surface 430 includes an elongated obround shape defined in a rectangular indent 436. The track 438 guides the paddle lead 122 through a first chamber 440 into a second chamber 442 having a port surface 444 tapering in diameter distally to a third chamber 446 matching the lumen 412 of the sheath 406. Stated differently, the size and profile shape of the third chamber 446 may match the size and profile shape of the lumen 412 at the proximal end 420 of the sheath 406. The varying profile shape of the insertion port 416 collapses the paddle lead 122 for transition from a flat paddle profile at the proximal surface 430 into a furled paddle profile for advancement through a profile shape of the proximal end 420 of the sheath 406.

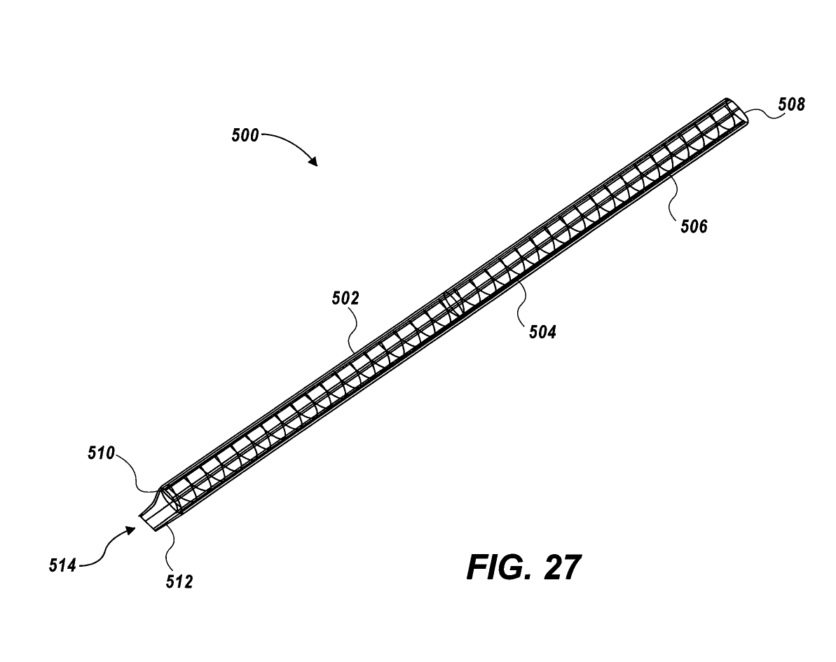

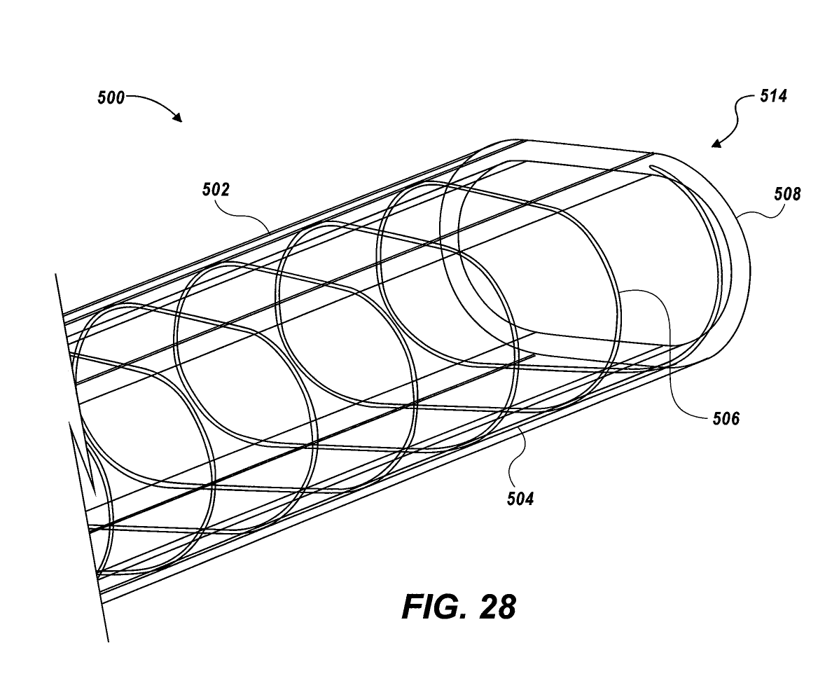

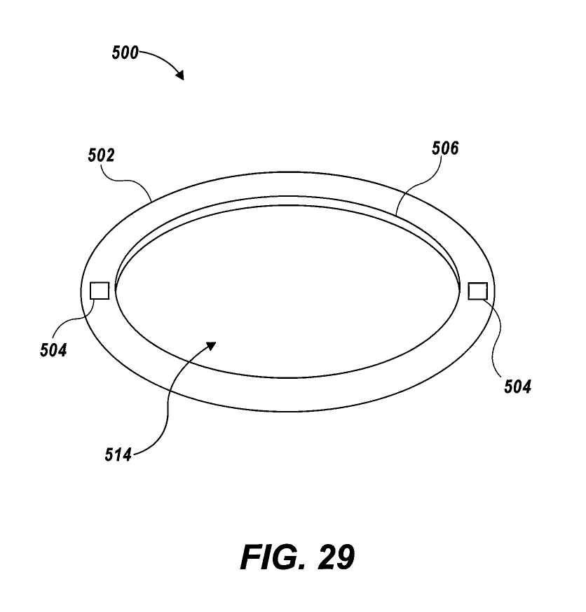

Turning to FIGS. 27-29, a detailed description of a sheath 500 with malleable spines 504 and an embedded coil 506 is provided. In one implementation, the sheath 500 includes an elongated body 502 extending between a proximal end 508 and a distal tip 510. A delivery stage 512 disposed near the distal tip 510 may include a distally extending or projecting lip, ledge, or the like to provide additional control of the deployment of the paddle lead 122. The sheath 500 includes a lumen 514 extending distally through a length of the sheath 500 from the proximal end 508 to the distal tip 510. The malleable spines 504 permit bending of the sheath 500 to an angle to maneuver through the patient anatomy to the target position in the epidural space 10, while the embedded coil 506 provides flexibility and prevents kinking.

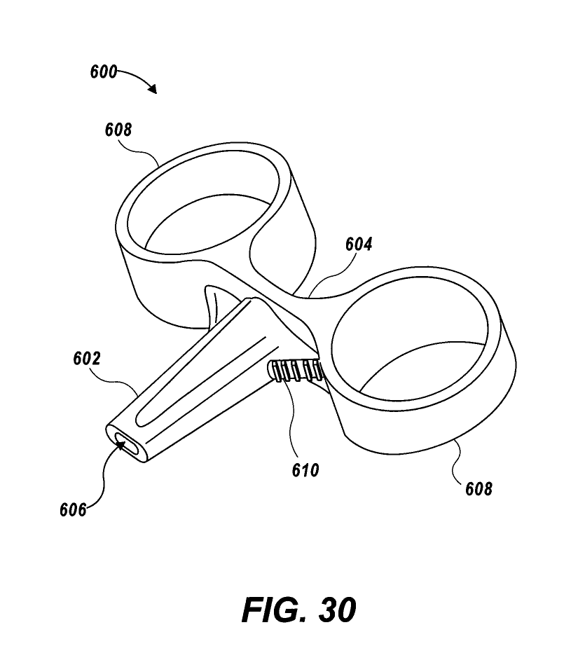

FIG. 30 shows an example hub 600 having a body 602 with an insertion port 606 extending through the body 602 distally from a proximal side 604. The hub 600 includes finger loops 608 and grips 610 to facilitate maneuvering during the procedure. It will be appreciated that various configurations of hubs 116 and sheaths 114 may be used to furl, unfurl, and deploy the paddle lead 122 without departing from the spirit and scope of the present disclosure.

Various other modifications and additions can be made to the exemplary implementations discussed without departing from the spirit and scope of the presently disclosed technology. For example, while the embodiments described above refer to particular features, the scope of this disclosure also includes implementations having different combinations of features and implementations that do not include all of the described features. Accordingly, the scope of the presently disclosed technology is intended to embrace all such alternatives, modifications, and variations together with all equivalents thereof.

What is claimed is:

1. A delivery tool for implanting a paddle lead, the delivery tool comprising:
a hub having a handle port and an insertion port;
a handle engaged to the handle port of the hub and extending proximally from the hub; and
a sheath extending distally from the hub, the sheath including a lumen extending through an elongated body from a proximal end to a distal tip, the insertion port including a port surface configured to collapse the paddle lead for passage into the lumen of the sheath.

2. The delivery tool of claim 1, wherein the engagement of the handle to the handle port of the hub permits movement of the handle to a plurality of positions.

3. The delivery tool of claim 2, wherein the handle moves to the plurality of positions by pivoting.

4. The delivery tool of claim 1, wherein the handle port of the hub is pivotally engaged to the handle.

5. The delivery tool of claim 1, wherein the handle terminates in a pivot ball at a distal end, the pivot ball engaged to the handle port and movable to a plurality of positions.

6. The delivery tool of claim 1, wherein the hub includes a proximal surface, the insertion port tapering distally in diameter from the proximal surface to a distal edge.

7. The delivery tool of claim 6, wherein the port surface transitions a first profile defined in the proximal surface to a second profile of the distal edge.

8. The delivery tool of claim 7, wherein the second profile of the distal edge matches a profile of the proximal end of the sheath.

9. The delivery tool of claim 1, wherein the sheath includes one or more profile shapes.

10. The delivery tool of claim 1, wherein the sheath is malleable.

11. The delivery tool of claim 1, wherein the sheath includes a fixed curve.

12. The delivery tool of claim 1, wherein the distal tip moves from a closed position to an open position as the paddle lead exits the sheath.

13. A delivery tool for implanting a paddle lead, the delivery tool comprising:
a hub having a body extending from a proximal surface to a distal surface;
a sheath receiver defined in the body of the hub;
a sheath engaged to the sheath receiver, the sheath extending distally from the hub, the sheath including a lumen extending through an elongated body from a proximal end to a distal tip; and
an insertion port extending through the body of the hub, the insertion port including a port surface configured to collapse the paddle lead for passage into the lumen of the sheath.

14. The delivery tool of claim 13, wherein the port surface extends distally from the proximal surface to a distal edge of the insertion port.

15. The delivery tool of claim 14, wherein the distal edge of the insertion port is coplanar with the proximal end of the sheath.

16. The delivery tool of claim 14, wherein the port surface extends distally at an angle.

17. The delivery tool of claim 14, wherein the port surface transitions the paddle lead from a first profile of the insertion port defined in the proximal surface of the hub to a second profile at the distal edge of the insertion port, the first profile being different than the second profile and the second profile matching a sheath profile of the proximal end of the sheath.

18. The delivery tool of claim 13, wherein the sheath is malleable.

19. The delivery tool of claim 13, wherein the sheath includes a fixed curve.

20. The delivery tool of claim 13, wherein the distal tip moves from a closed position to an open position as the paddle lead exits the sheath.

21. The delivery tool of claim 13, wherein the sheath includes one or more profile shapes.

22. The delivery tool of claim 13, further comprising:
a handle port defined in the body of the hub; and
a handle engaged to the handle port and extending proximally from the hub.

23. The delivery tool of claim 22, wherein the handle port of the hub is pivotally engaged to the handle.

24. A method of implanting a paddle lead, the method comprising:
receiving the paddle lead at a first profile of an insertion port extending from a proximal surface of a hub to a distal edge of the hub, the first profile defined in the proximal surface;
collapsing the paddle lead using a port surface of the insertion port, the port surface transitioning the paddle lead from the first profile to a second profile at the distal edge, the first profile being different than the second profile and the second profile matching a sheath profile of a proximal end of a sheath, the sheath having a lumen extending from the proximal end to a distal tip; and
advancing the paddle lead through the lumen of the sheath through the distal tip.
